(19) United States
(12) Patent Application Publication
(10) Pub. No.: US 2006/0014969 A1
(43) Pub. Date: Jan. 19, 2006

(54) Method for the continuous intermediate separation of an oxirane produced by the oxirane synthesis with no coupling product by means of a partition-wall column

(76) Inventors: Peter Bassler, Viernheim (DE); Hans-Georg Gobbel, Kallstadt (DE); Joaquim Henrique Teles, Otterstadt (DE); Peter Rudolf, Ladenburg (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/521,790

(22) PCT Filed: Jul. 22, 2003
(86) PCT No.: PCT/EP03/07989

§ 371 Date: Jul. 27, 2005

(30) Foreign Application Priority Data
Jul. 23, 2002 (DE) 10233383.1

(51) Int. Cl. C07D 301/19

(52) U.S. Cl. 549/525

(57) ABSTRACT

Continuously operated process for the intermediate isolation of the oxirane formed by reaction of a hydroperoxide with an organic compound in the oxirane synthesis, wherein the product mixture formed in the synthesis is fractionated in a dividing wall column to give a low-boiling fraction, an intermediate-boiling fraction and a high-boiling fraction and the oxirane is taken off in the intermediate-boiling fraction at the side offtake and the hydroperoxide is taken off in the high-boiling fraction at the bottom of the column.

The present invention relates to a continuously operated process for the intermediate isolation of the oxirane formed by reaction of a hydroperoxide with an organic compound in the preferably coproduct-free oxirane synthesis, wherein the product mixture formed in the synthesis is fractionated in a dividing wall column to give a low-boiling fraction, an intermediate-boiling fraction and a high-boiling fraction and the oxirane is taken off in the intermediate-boiling fraction at the side offtake of the column and the hydroperoxide is taken off in the high-boiling fraction at the bottom of the column.

In current processes of the prior art, oxiranes can be prepared by reaction of suitable organic compounds with hydroperoxides in one or more stages.

For example, the multistage process described in WO 00/07965 provides for the reaction of the organic compound with a hydroperoxide to comprise at least the steps (i) to (iii):
(i) reacting the hydroperoxide with the organic compound to give a product mixture comprising the reacted organic compound and unreacted hydroperoxide,
(ii) separating the unreacted hydroperoxide from the mixture resulting from step (i),
(iii) reacting the hydroperoxide which has been separated off in step (ii) with the organic compound.

Accordingly, the reaction of the organic compound with the hydroperoxide takes place in at least two steps (i) and (iii), with the hydroperoxide separated off in step (ii) being reused in the reaction.

The reactions in steps (i) and (iii) are preferably carried out in two separate reactors, preferably fixed-bed reactors, with the reaction of step (i) preferably taking place in an isothermal reactor and the reaction of step (iii) taking place in an adiabatic reactor.

In general, this multistage process can be used for reacting alkenes with hydroperoxides to form oxiranes. The hydroperoxide used in this sequence is preferably hydrogen peroxide and the organic compound is preferably brought into contact with a heterogeneous catalyst during the reaction.

The above process can, in particular, be used for preparing propylene oxide from propylene and hydrogen peroxide. The reaction is preferably carried out in methanol as solvent. The propylene used is usually “chemical grade” propylene and contains about 4% by weight of propane. The hydrogen peroxide conversion in step (i) is from about 85% to 90% and that in step (iii) is about 95%, based on step (ii). Over the two steps, a hydrogen peroxide conversion of about 99% can be achieved at a propylene oxide selectivity of about 94-95%.

Owing to the high selectivity of the reaction, this synthesis is also referred to as a coproduct-free oxirane synthesis.

In this process, the separation of, in particular, the hydroperoxide from the remainder of the product mixture has been optimized, since the unreacted hydroperoxide from step (i) is to be reused in the reaction. The hydroperoxide is preferably separated off by distillation, where it is taken off at the bottom of a column.

The oxirane can be separated off directly from the product mixture in the same column as the hydroperoxide. In this intermediate isolation by distillation, the oxirane is then taken from the mixture via the top of the column. The term “intermediate isolation” refers to the separation of the oxirane directly from the reaction mixture, in contrast to the purification by distillation which is carried out on the oxirane which has previously been separated off.

If propylene is reacted with hydrogen peroxide, the reaction mixture to be fractionated by distillation comprises, for example, methanol, water, propylene oxide as oxirane, byproducts such as methoxypropanols, 1,2-propylene diglycol, acetaldehyde, methyl formate, unreacted propylene as organic compound, propane and hydrogen peroxide as hydroperoxide.

The oxirane distilled off via the top of the column according to the prior art is contaminated with compounds among those listed above which behave as volatile low boilers under the distillation conditions, for example with unreacted organic compound. It then normally has to be subjected to a further purification step, i.e. at purification by distillation. This can be carried out in a further distillation column which is connected in series to the column used as separation apparatus. This procedure involving at least two-fold distillation of the desired product requires an increased outlay in terms of apparatus and energy.

It is an object of the present invention to optimize the separation by distillation of the oxiranes formed in the reaction of suitable organic compounds with hydroperoxides from the reaction mixture, in particular so as to provide a process which is improved in respect of the energy consumption in the distillation and the thermal stress to which the products are subjected. In particular, it is an object to provide a process which is operated continuously and allows the oxiranes obtained, preferably by multistage reaction, to be isolated in high purity by intermediate isolation with a low outlay in terms of apparatus and energy.

We have found that this object is achieved by a continuously operated process for the intermediate isolation by means of a dividing wall column of the oxirane formed by reaction of a hydroperoxide with an organic compound in the preferably coproduct-free oxirane synthesis.

The present invention accordingly provides a continuously operated process for the intermediate isolation of the oxirane formed by reaction of a hydroperoxide with an organic compound in the oxirane synthesis, wherein the product mixture formed in the synthesis is fractionated in a dividing wall column to give a low-boiling fraction, an intermediate-boiling fraction and a high-boiling fraction and the oxirane is taken off in the intermediate-boiling fraction at the side offtake and the hydroperoxide is taken off in the high-boiling fraction at the bottom of the column.

The process of the present invention allows the oxirane to be isolated directly from the reaction mixture by intermediate isolation by distillation in the same column in which the hydroperoxide is separated off by distillation. Furthermore, the oxirane and the hydroperoxide used can be separated from one another under mild conditions and with little thermal stress in the process of the present invention, since only short residence times are necessary in the dividing wall column compared to two columns connected in series. This is extremely advantageous since both compounds are highly reactive and thermally labile components. Compared to the method disclosed in the prior art, the novel process of the present invention therefore leads to a reduced outlay in terms of apparatus and energy combined with an improved product quality. Furthermore, the dividing wall column has a particularly low energy consumption and thus offers advantages in respect of the energy requirement compared to a conventional column. This is extremely advantageous for industrial use.

Distillation columns having side offtakes and a dividing wall, hereinafter also referred to as dividing wall columns, are known. They represent a development of distillation columns which have a side offtake but no dividing wall. The possible uses of the last-named type of column are, however, restricted because the products taken off at the side offtakes are never completely pure. In the case of products taken off at the side in the enrichment section of the column, which are usually in liquid form, the side product still contains proportions of low-boiling components which should be separated off via the top. In the case of products taken off at the side in the stripping section of the column, which are usually in vapor form, the side product still contains proportions of high boilers. The use of conventional side offtake columns is therefore restricted to cases in which contaminated side products are permissible.

However, when a dividing wall is installed in such a column, the effectiveness of the separation can be improved. In this type of construction, it is possible to take off side products in pure form. In the middle region above and below the feed point and the side offtake, there is a dividing wall which may be welded in place or may only be physically locked in position. It seals the offtake part from the inflow part and suppresses cross-mixing of liquid and vapor streams over the entire column cross section in this part of the column. In the case of multicomponent mixtures whose components have similar boiling points, this reduces the total number of distillation columns required.

This type of column has been used, for example, for separating a mixture of methane, ethane, propane and butane (U.S. Pat. No. 2,471,134), for separating a mixture of benzene, toluene and xylene (U.S. Pat. No. 4,230,533) and for separating a mixture of n-hexane, n-heptane and n-octane (EP 0 122 367).

Dividing wall columns can also be used successfully for separating azeotropically boiling mixtures (EP 0 133 510).

Finally, dividing wall columns in which chemical reactions can be carried out with simultaneous distillation of the products are also known. Examples of such reactions are esterifications, transesterifications, saponifications and acetalizations (EP 0 126 288).

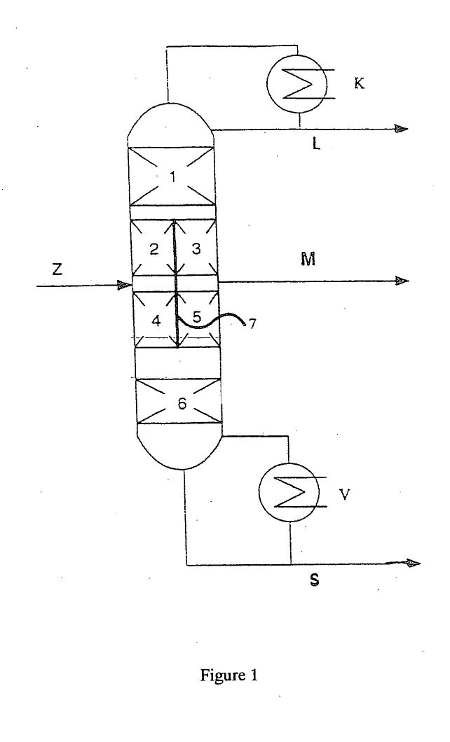

FIG. 1 schematically shows the intermediate separation of the oxirane formed in the oxirane synthesis from the hydroperoxide used in excess in a dividing wall column. Here, the reaction mixture coming from the oxirane synthesis is introduced into the column as feed Z. In the column, this reaction mixture is fractionated to give a low-boiling fraction L which consists essentially of unreacted organic compound, an intermediate-boiling fraction comprising the oxirane and a high-boiling fraction S which consists essentially of unreacted hydroperoxide together with solvent and water.

The oxirane is taken off at the side offtake for intermediate boilers M. The organic compound used can be isolated from the low-boiling fraction which distills off at the top of the column and can be reacted once again with hydroperoxide in the apparatuses provided for this purpose.

The intermediate-boiling fraction comprising the oxirane as desired product is taken off in liquid or vapor form at the side offtake. To take off this fraction at the side offtake, it is possible to use receivers which are located either inside or outside the column and in which the liquid or condensing vapor can be collected.

The high-boiling fraction, which typically comprises the hydroperoxide together with the solvent used and water and is taken off at the bottom of the column, can be reacted once again with the organic compound in the apparatuses provided for this purpose.

The oxirane synthesis is preferably carried out using the process and the apparatus for carrying out the process described in WO 00/07965. The apparatus comprises an isothermal fixed-bed reactor, a separation apparatus and an adiabatic fixed-bed reactor.

Use of the dividing wall column of the process of the present invention as separation apparatus makes it possible to have a plant by means of which the oxirane can be prepared continuously and be isolated by continuous intermediate isolation and unreacted starting materials can be returned to the oxirane synthesis. In a first stage, the organic compound is reacted with the hydroperoxide in an isothermal reactor and the reaction mixture is transferred to the dividing wall column where the oxirane is obtained in the intermediate-boiling fraction taken from the side offtake and the hydroperoxide is obtained in the high-boiling fraction. The latter compound is then reacted once again with the organic compound in a second stage in an adiabatic reactor.

If, for example, propylene is used as organic compound, this can also be used as starting material recovered via the top of the column.

Accordingly, the process of the present invention is particularly useful for the continuous intermediate isolation of an oxirane from a product mixture which is prepared by a process comprising at least the steps (i) to (iii):
(i) reacting the hydroperoxide with the organic compound to give a product mixture comprising the reacted organic compound and unreacted hydroperoxide,
(ii) separating the unreacted hydroperoxide from the mixture resulting from step (i),
(iii) reacting the hydroperoxide which has been separated off in step (ii) with the organic compound.

To carry out the process of the present invention, it is possible to use customary dividing wall columns having one or more side offtakes, for example columns as are mentioned in the prior art.

Such a dividing wall column has, for example, preferably from 10 to 70, more preferably from 15 to 50, particularly preferably from 20 to 40, theoretical plates. The process of the present invention can be carried out particularly advantageously using this configuration.

In this type of column, the upper combined region 1 of the inflow part and offtake part of the dividing wall column preferably has from 5 to 50%, more preferably from 15 to 30%, of the total number of theoretical plates in the column, the enrichment section 2 of the inflow part preferably has from 5 to 50%, more preferably from 15 to 30%, the stripping section 4 of the inflow part preferably has from 5 to 50%, more preferably from 15 to 30%, the stripping section 3 of the offtake part preferably has from 5 to 50%, more preferably from 15 to 30%, the enrichment section 5 of the offtake part preferably has from 5 to 50%, more preferably from 15 to 30%, and the combined lower region 6 of the feed part and offtake part preferably has from 5 to 50%, more preferably from 15 to 30%. The sum of the number of theoretical plates in the regions 2 and 4 in the inflow part is preferably from 80 to 110%, more preferably from 90 to 100%, of the sum of the number of theoretical plates in the regions 3 and 5 in the offtake part.

It is likewise favorable to locate the feed point and the offtake at different heights in the column relative to the position of the theoretical plates. The feed point is preferably located at a position which is from 1 to 8, more preferably from 3 to 5, theoretical plates above or below the side offtake.

The dividing wall column used in the process of the present invention is preferably configured either as a packed column containing random packing elements or ordered packing or as a tray column. For example, sheet metal or mesh packing having a specific surface area of from 100 to 1000 m²/m³, preferably from about 250 to 750 m²/m³, can be used as ordered packing. Such packing offers a high separation performance combined with a low pressure drop per theoretical plate.

In the abovementioned configuration of the column, the region of the column which is divided by the dividing wall 7 and comprises the enrichment section 2 of the inflow part, the stripping section 3 of the offtake part, the stripping section 4 of the inflow part and the enrichment section 5 or parts thereof is preferably provided with ordered packing or random packing elements and the dividing wall is thermally insulated in these regions.

The mixture obtained in the oxirane synthesis which comprises low boilers L, intermediate boilers and high boilers S, is then introduced continuously into the column as feed Z. This feed stream is generally liquid. However, it can be advantageous to subject the feed stream to prevaporization and subsequently introduce it into the column as a two-phase, i.e. gaseous and liquid, stream or in the form of a gaseous stream and a liquid stream. This prevaporization is particularly useful when the feed stream contains relatively large amounts of low boilers L. The prevaporization can significantly reduce the load on the stripping section of the column.

The feed stream is advantageously introduced in a quantity-regulated manner into the inflow part by means of a pump or via a static inflow head of at least 1 m. This addition is preferably carried out via a cascade regulation in combination with the liquid level regulation of the accommodation space of the inflow part. The regulation is set so that the amount of liquid introduced into the enrichment section 2 cannot drop below 30% of the normal value. It has been found that such a procedure is important to even out troublesome fluctuations in the feed flow or the feed concentration.

It is similarly important for the division of the liquid flowing down from the stripping section 3 of the offtake part of the column between the side offtake and the enrichment section 5 of the offtake part to be set by means of a regulating facility so that the amount of liquid introduced into the region 5 cannot drop below 30% of the normal value.

Adherence to these requirements has to be ensured by appropriate regulation methods.

Regulation mechanisms for operating dividing wall columns have been described, for example, in Chem. Eng. Technol. 10 (1987) 92-98, Chem.-Ing.-Technol. 61 (1989) No. 1, 16-25, Gas Separation and Purification 4 (1990) 109-114, Process Engineering 2 (1993) 33-34, Trans IChemE 72 (1994) Part A 639-644, Chemical Engineering 7 (1997) 72-76. The regulation mechanisms described in this prior art can also be employed for the process of the present invention or be applied thereto.

The regulation principle described below has been found to be particularly useful for the continuously operated intermediate separation of the oxirane from the hydroperoxide used in excess. It is readily able to cope with fluctuations in loading. The distillation is thus preferably taken off under temperature control.

A temperature regulation device which utilizes the downflow quantity, the reflux ratio or preferably the quantity of runback as regulating parameter is provided in the upper section 1 of the column. The measurement point for the temperature regulation is preferably located from 3 to 8, more preferably from 4 to 6, theoretical plates below the upper end of the column.

Appropriate setting of the temperature then results in the liquid flowing down from the section 1 of the column being divided at the upper end of the dividing wall so that the ratio of the liquid flowing to the inflow part to that flowing to the offtake part is preferably from 0.1 to 1.0, more preferably from 0.3 to 0.6.

In this method, the downflowing liquid is preferably collected in a receiver which is located in or outside the column and from which the liquid is then fed continuously into the column. This receiver can thus take on the task of a pump reservoir or provide a sufficiently high static head liquid which makes it possible for the liquid to be passed on further in a regulated manner by means of regulating devices, for example valves. When packed columns are used, the liquid is firstly collected in collectors and from there conveyed to an internal or external receiver.

The vapor stream at the lower end of the dividing wall is set by selection and/or dimensioning of the separation internals and/or incorporation of pressure-reducing devices, for example orifice plates, so that the ratio of the vapor stream in the inflow part to that in the offtake part is preferably from 0.8 to 1.2, preferably from 0.9 to 1.1.

In the abovementioned regulation principle, a temperature regulation device which utilizes the quantity taken off at the bottom as regulating parameter is provided in the lower combined section 6 of the column. The bottom product can therefore be taken off under temperature control. The measurement point for the temperature regulation device is preferably located from 3 to 6, more preferably from 4 to 6, theoretical plates above the lower end of the column.

In addition, the level regulation in column section 6 and thus for the bottom of the column can be utilized for regulating the quantity taken off at the side offtake. For this purpose, the liquid level in the vaporizer is used as regulating parameter.

The differential pressure over the column can also be utilized as regulating parameter for the heating power. The distillation is advantageously carried out at a pressure at the top of from 0.5 to 5 bar, preferably from 0.7 to 2 bar. Accordingly, the heating power of the vaporizer at the bottom of the column is selected to maintain this pressure range.

The distillation temperature resulting therefrom is preferably from 10 to 60° C., more preferably from 25 to 45° C. It is measured at the side offtake.

Accordingly, the pressure at the top of the dividing wall column in a preferred embodiment of the process of the present invention is from 0.5 to 5 bar.

Furthermore, the distillation temperature at the side offtake in a preferred embodiment of the process of the present invention is from 10 to 60° C.

To be able to operate the dividing wall column in a trouble-free manner, the abovementioned regulation mechanisms are usually employed in combination.

In the separation of multicomponent mixtures into low-boiling, intermediate-boiling and high-boiling fractions, there are usually specifications in respect of the maximum permissible proportion of low boilers and high boilers in the middle fraction. Here, individual components which are critical to the separation problem, referred to as key components, or else the sum of a plurality of key components are/is specified.

Adherence to the specification for the high boilers in the intermediate-boiling fraction is preferably regulated via the division ratio of the liquid at the upper end of the dividing wall. The division ratio is set so that the concentration of key components for the high-boiling fraction in the liquid at the upper end of the dividing wall amounts to from 10 to 80% by weight, preferably from 30 to 50% by weight, of the value which is to be achieved in the stream taken off at the side. The liquid division can then be set so that when the concentration of key components of the high-boiling fraction is higher, more liquid is introduced into the inflow section, and when the concentration of key components is lower, less liquid is introduced into the inflow section.

Accordingly, the specification for the low boilers in the intermediate-boiling fraction is regulated by means of the heating power. Here, the heating power in the vaporizer is set so that the concentration of key components for the low-boiling fraction in the liquid at the lower end of the dividing wall amounts to from 10 to 80% by weight, preferably from 30 to 50% by weight, of the value which is to be achieved in the product taken off at the side. Thus, the heating power is set so that when the concentration of key components of the low-boiling fraction is higher, the heating power is increased, and when the concentration of key components of the low-boiling fraction is lower, the heating power is reduced.

The concentration of low and high boilers in the intermediate-boiling fraction can be determined by customary analytical methods. For example, infrared spectroscopy can be used for detection, with the compounds present in the reaction mixture being identified by means of their characteristic absorptions. These measurements can be carried out in-line directly in the column. However, preference is given to using gas-chromatographic methods. In this case, sampling facilities are then provided at the upper and lower end of the dividing wall. Liquid or gaseous samples can then be taken continuously or at intervals from the column and analyzed to determine their compositions. The appropriate regulation mechanisms can then be activated as a function of the composition.

It is an objective of the process of the present invention to provide oxiranes having a purity of preferably at least 95%, but particularly preferably at least 97%, with the sum of oxirane and or the components present in the oxirane being 100% by weight.

In a specific embodiment of the dividing wall column, it is also possible for the inflow part and offtake part which are separated from one another by the dividing wall 7 not to be present in one column but to be physically separate from one another. In this specific embodiment, the dividing wall column can thus comprise at least two physically separate columns which then have to be thermally coupled with one another. Such thermally coupled columns exchange vapor and liquid between them, but energy is introduced via only one column. This specific embodiment has the advantage that the thermally coupled columns can also be operated under different pressures, which can make it possible to achieve better setting of the temperature level required for the distillation than in the case of a conventional dividing wall column.

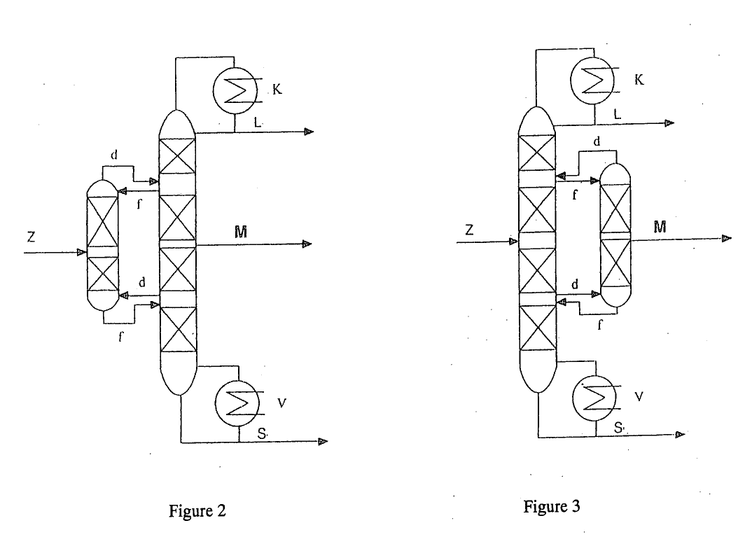

Examples of dividing wall columns in the specific embodiment of thermally coupled columns are shown schematically in FIGS. 2 and 3.

FIG. 2 shows a variant in which energy is introduced via the vaporizer V of the column which is located downstream of the column into which the product mixture is fed as feed Z. In this arrangement, the product mixture is firstly separated into a low-boiling fraction and a high-boiling fraction, each of which also contain intermediate boilers, in the first column. The resulting fractions are subsequently transferred to the second column, with the low-boiling fraction comprising intermediate boilers being fed at the upper end of the second column and the high-boiling fraction comprising intermediate boilers being fed in at the lower end. The low boilers L are distilled off via the top of the column and isolated via the condenser K. The high boilers S are obtained in the bottoms from the column. The purified propylene oxide can be taken off at the side offtake for intermediate boilers M. The two columns can exchange vapor and liquid via d and f.

FIG. 3 shows a further variant of thermally coupled columns. In this embodiment, the energy is introduced via the vaporizer V of the column into which the reaction mixture is also fed as feed Z. The low boilers L are distilled off via the top of this column and are condensed by means of the condenser K. The high boilers S are obtained in the bottoms. Low boilers L enriched with intermediate boilers are then transferred to the upper part of the downstream column and high boilers S enriched with intermediate boilers are transferred to the lower part of the downstream column. The purified propylene oxide can be taken off from the side offtake for intermediate boilers M. The two columns can exchange vapor and liquid via d and f.

The columns of FIGS. 2 and 3 can also be configured as packed columns containing random packing or ordered packing or as tray columns. For example, sheet metal or mesh packing having a specific surface area of from 100 to 1000 m²/m³, preferably from about 250 to 750 m 2/m³, can be used as ordered packing. Such packing provides a high separation efficiency combined with a low pressure drop per theoretical plate.

If the process of the present invention is employed for intermediate isolation of propylene oxide, the propylene oxide should be obtained in a purity of preferably at least 95% by weight. The concentration of key components of the low boilers (e.g. acetaldehyde, methyl formate) and of key components of the high boilers (e.g. methanol, water, propylene glycol) in the product should then preferably be less than 5% by weight, with the sum of oxirane and key components being 100% by weight.

Therefore, the present invention also relates to a process as described above wherein the sum of key components in the purified oxirane is less than 5% by weight, with the sum of oxirane and all the other components present in the oxirane being 100% by weight.

For the process of the present invention for the continuously operated intermediate isolation in a dividing wall column of the oxirane formed in the coproduct-free oxirane synthesis, it is possible to use the starting materials known from the prior art for the oxirane synthesis.

Preference is given to using organic compounds which have at least one C-C double bond. Examples of such organic compounds having at least one C-C double bond include the following alkenes:

ethene, propylene, 1-butene, 2-butene, isobutene, butadiene, pentenes, piperylene, hexenes, hexadienes, heptenes, octenes, diisobutene, trimethylpentene, nonenes, dodecene, tridecene, tetradecene to eicosene, tripropene and tetrapropene, polybutadienes, polyisobutenes, isoprene, terpenes, geraniol, linalool, linalyl acetate, methylenecyclopropane, cyclopentene, cyclohexene, norbornene, cycloheptene, vinylcyclohexane, vinyloxirane, vinylcyclohexene, styrene, cyclooctene, cyclooctadiene, vinylnorbornene, indene, tetrahydroindene, methylstyrene, dicyclopentadiene, divinylbenzene, cyclododecene, cyclododecatriene, stilbene, diphenylbutadiene, vitamin A, beta-carotene, vinylidene fluoride, allyl halides, crotyl chloride, methallyl chloride, dichlorobutene, allyl alcohol, methallyl alcohol, butenols, butenediols, cyclopentenediols, pentenols, octadienols, tridecenols, unsaturated steroids, ethoxyethene, isoeugenol, anethole, unsaturated carboxylic acids such as acrylic acid, methacrylic acid, crotonic acid, maleic acid, vinylacetic acid, unsaturated fatty acids such as oleic acid, linoleic acid, palmitic acid, naturally occurring fats and oils.

Preference is given to alkenes having from 2 to 8 carbon atoms. Particular preference is given to reacting ethene, propylene and butene. Very particular preference is given to reacting propylene.

As hydroperoxide, it is possible to use the known hydroperoxides which are suitable for the reaction with the organic compound. Examples of such hydroperoxides are tert-butyl hydroperoxide and ethylbenzene hydroperoxide. Preference is given to using hydrogen peroxide as hydroperoxide for the oxirane synthesis, with an aqueous hydrogen peroxide solution also being able to be used.

The preparation of hydrogen peroxide can be carried out using, for example, the anthraquinone process by means of which virtually the entire world production of hydrogen peroxide is produced. This process is based on the catalytic hydrogenation of an anthraquinone compound to form the corresponding anthrahydroquinone compound, subsequent reaction of this with oxygen to form hydrogen peroxide and subsequent extraction to separate off the hydrogen peroxide formed. The catalysis cycle is closed by renewed hydrogenation of the anthraquinone compound which is obtained back.

An overview of the anthraquinone process is given in “Ullmann's Encyclopedia of Industrial Chemistry”, 5th Edition, Volume 13, pages 447 to 456.

It is likewise conceivable to obtain hydrogen peroxide by converting sulfuric acid into peroxodisulfuric acid by anodic oxidation with simultaneous evolution of hydrogen at the cathode. Hydrolysis of the peroxodisulfuric acid then leads via peroxomonosulfuric acid to hydrogen peroxide and sulfuric acid, which is thus recovered.

It is of course also possible to prepare hydrogen peroxide from the elements.

In the individual reactors, it is possible, when the organic compound is selected appropriately, for the reaction of this with the hydroperoxide to occur under the prevailing pressure and temperature conditions without addition of catalyst.

However, preference is given to a process in which one or more suitable catalysts are added to increase the efficiency of the reaction; once again, heterogeneous catalysts are preferably used.

All heterogeneous catalysts which are suitable for the respective reaction are conceivable. Preference is given to using catalysts which comprise a porous oxidic material, e.g. a zeolite. The catalysts used preferably comprise a titanium-, germanium-, tellurium-, vanadium, chromium-, niobium- or zirconium-containing zeolite as porous oxidic material.

Specific mention may be made of titanium-, germanium-, tellurium-, vanadium-, chromium-, niobium- and zirconium-containing zeolites having a pentasil zeolite structure, in particular the types which can be assigned X-ray-crystallographically to the ABW, ACO, AEI, AEL, AEN, AET, AFG, AFI, AFN, AFO, AFR, AFS, AFT, AFX, AFY, AHT, ANA, APC, APD, AST, ATN, ATO, ATS, ATT, ATV, AWO, AWW, BEA, BIK, BOG, BPH, BRE, CAN, CAS, CFI, CGF, CGS, CHA, CHI, CLO, CON, CZP, DAC, DDR, DFO, DFT, DOH, DON, EAB, EDI, EMT, EPI, ERI, ESV, EUO, FAU, FER, GIS, GME, GOO, HEU, IFR, ISV, ITE, JBW, KFI, LAU, LEV, LIO, LOS, LOV, LTA, LTL, LTN, MAZ, MEI, MEL, MEP, MER, MFI, MFS, MON, MOR, MSO, MTF, MTN, MTF, MTW, MWW, NAT, NES, NON, OFF, OSI, PAR, PAU, PHI, RHO, RON, RSN, RTE, RTH, RUT, SAO, SAT, SBE, SBS, SBT, SFF, SGT, SOD, STF, STI, STT, TER, THO, TON, TSC, VET, VFI, VNI, VSV, WIE, WEN, YUG, ZON structure or to mixed structures comprising two or more of the abovementioned structures. Furthermore, titanium-containing zeolites having the ITQ-4, SSZ-24, TFM-1, UTD-1, CIT-1 or CIT-5 structure are also conceivable for use in the process of the present invention. Further titanium-containing zeolites which may be mentioned are those of the ZSM-48 or ZSM-12 structure.

Particular preference is given to Ti zeolites having an MFI or MEL structure or an MFI/MEL mixed structure. Very particular preference is given to the titanium-containing zeolite catalysts which are generally referred to as “TS-1”, “TS-2”, “TS-3” and also Ti zeolites having a framework structure isomorphous with 3-zeolite.

In particular, it is advantageous to use a heterogeneous catalyst comprising the titanium-containing silicalite TS-1.

It is possible to use the porous oxidic material itself as catalyst. However, it is also of course possible for the catalyst used to be a shaped body comprising the porous oxidic material. All processes known from the prior art can be used for producing the shaped body from the porous oxidic material.

Noble metals in the form of suitable noble metal components, for example in the form of water-soluble salts, can be applied to the catalyst material before, during or after the one or more shaping steps in these processes. This method is preferably employed for producing oxidation catalysts based on titanium silicates or vanadium silicates having a zeolite structure, and it is thus possible to obtain catalysts which contain from 0.01 to 30% by weight of one or more noble metals from the group consisting of ruthenium, rhodium, palladium, osmium, iridium, platinum, rhenium, gold and silver. Such catalysts are described, for example, in DE-A 196 23 609.6.

The shaped bodies can also be processed further. All methods of comminution are conceivable, for example splitting or crushing the shaped bodies, as are further chemical treatments as are described above by way of example.

When a shaped body or a plurality thereof is used as catalyst, it/they can, after deactivation has occurred in the process of the present invention, be regenerated by a method in which the deposits responsible for deactivation are burned off in a targeted manner. This is preferably carried out in an inert gas atmosphere containing precisely defined amounts of oxygen-donating substances. This regeneration process is described in DE-A 197 23 949.8. It is also possible to use the regeneration processes mentioned there in the discussion of the prior art.

As solvents, it is possible to use all solvents which completely or at least partly dissolve the starting materials used in the oxirane synthesis. Examples of solvents are aliphatic, cycloaliphatic and aromatic hydrocarbons, esters, ethers, amides, sulfoxides and ketones and also alcohols. The solvents can also be used in the form of mixtures. Preference is given to using alcohols. The use of methanol as solvent is particularly preferred.

As reactors for the oxirane synthesis, it is of course possible to use all conceivable reactors which are best suited to the respective reactions. A reactor is not restricted to an individual vessel for the oxirane synthesis. Rather, it is also possible to use, for example, a cascade of stirred vessels.

Fixed-bed reactors are preferably used as reactors for the oxirane synthesis. Further preference is given to using fixed-bed tube reactors as fixed-bed reactors.

In the above-described oxirane synthesis which is preferably employed, particular preference is given to using an isothermal fixed-bed reactor as reactor for step (i) and an adiabatic fixed-bed reactor for step (iii).

The oxiranes used for the process of the present invention are thus preferably prepared in an isothermal fixed-bed reactor and an adiabatic fixed-bed reactor, with the intermediate isolation being carried out in a dividing wall column.

It is also possible to react a plurality of organic compounds with the hydroperoxide. It is likewise conceivable to use a plurality of hydroperoxides for the reaction. If, for example, two organic compounds and/or a plurality of hydroperoxides are reacted with one another in the respective steps, various products resulting from the reactions can be present in the mixtures. However, such mixtures of two different oxiranes can also be separated successfully in the process of the present invention by intermediate isolation by distillation using a dividing wall column having two side offtakes, as long as the boiling points are not too close together.

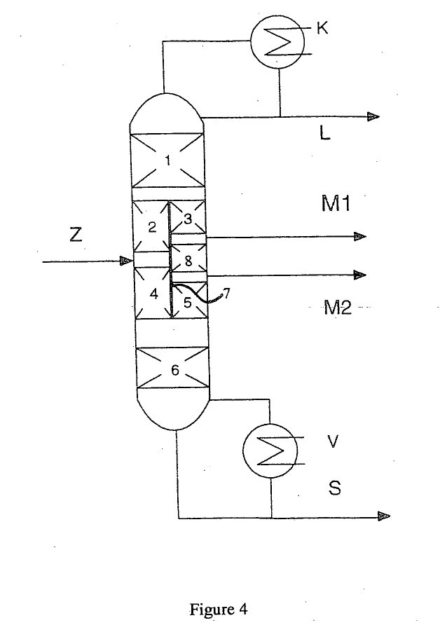

A dividing wall column having two side offtakes is shown schematically in FIG. 4. Here, the lower-boiling oxirane is taken off at the upper side offtake M1 and the higher-boiling oxirane is taken off at the lower side offtake M2. In this arrangement, the region of thermal coupling 8 preferably has from five to fifty percent, more preferably from fifteen to thirty percent, of the total number of theoretical plates in the column.

The invention further provides an apparatus for carrying out a continuously operated process for the intermediate isolation of the oxirane formed in the oxirane synthesis by reaction of a hydroperoxide with an organic compound.

In a preferred embodiment of an apparatus for carrying out a continuously operated process for the intermediate isolation of the oxirane formed in the oxirane synthesis by reaction of a hydroperoxide with an organic compound, the apparatus for preparing the oxirane comprises at least one isothermal reactor and one adiabatic reactor for carrying out the steps (i) and (iii) and a separation apparatus for the step (ii), where the separation apparatus comprises a dividing wall column having one or two side offtakes or at least two thermally coupled columns.

List of reference numerals for FIGS. 1, 2, 3 and 4:
1 Combined region of the inflow and offtake part of the dividing wall column
2 Enrichment section of the inflow part
3 Stripping section of the offtake part
4 Stripping section of the inflow part
5 Enrichment section of the offtake part
6 Combined region of the inflow and offtake part
7 Dividing wall
Z Feed
L Low boilers
M Side offtake for intermediate boilers
M1 Side offtake for lower-boiling oxirane
M2 Side offtake for higher-boiling oxirane
S High boilers
K Condenser
V Vaporizer
d Vapor
f Liquid

Horizontal and diagonal or indicated diagonal lines in the columns symbolize packing made up of random packing elements or ordered packing which may be present in the column.

1-10. (canceled)

11. A continuously operated process for the intermediate isolation of the oxirane formed by reaction of a hydroperoxide with an organic compound in the oxirane synthesis, wherein the product mixture formed in the synthesis is fractionated in a dividing wall column to give a low-boiling fraction, an intermediate-boiling fraction and a high-boiling fraction and the oxirane is taken off in the intermediate-boiling fraction at the side offtake and the hydroperoxide is taken off in the high-boiling fraction at the bottom of the column.

12. The process as claimed in claim 11, wherein the dividing wall column comprises at least two thermally coupled distillation columns.

13. The process as claimed in claim 11, wherein the dividing wall column has from 10 to 70 theoretical plates.

14. The process as claimed in claim 11, wherein the pressure at the top of the dividing wall column is from 0.5 to 5 bar and the distillation temperature at the side offtake is from 10 to 60° C.

15. The process as claimed in claim 11, wherein the dividing wall column has from 10 to 70 theoretical plates and wherein the pressure at the top of the dividing wall column is from 0.5 to 5 bar and the distillation temperature at the side offtake is from 10 to 60° C.

16. The process as claimed in claim 11, wherein the sum of key components in the purified oxirane is less than 5% by weight, with the sum of oxirane and all the other components present in the oxirane being 100% by weight.

17. The process as claimed in claim 11, wherein the product mixture comprising the oxirane is prepared by a process comprising at least the steps (i) to (iii):
(i) reacting the hydroperoxide with the organic compound to give a product mixture comprising the reacted organic compound and unreacted hydroperoxide,
(ii) separating the unreacted hydroperoxide from the mixture, as defined in claim 1, resulting from step (i),
(iii) reacting the hydroperoxide which has been separated off in step (ii) with the organic compound,
with an isothermal fixed-bed reactor being used in step (i) and an adiabatic fixed-bed reactor being used in step (iii).

18. The process as claimed in claim 17, wherein the dividing wall column has from 10 to 70 theoretical plates and wherein the pressure at the top of the dividing wall column is from 0.5 to 5 bar and the distillation temperature at the side offtake is from 10 to 60° C., and wherein the sum of key components in the purified oxirane is less than 5% by weight, with the sum of oxirane and all the other components present in the oxirane being 100% by weight.

19. The process as claimed in claim 11, wherein the hydroperoxide used is hydrogen peroxide and the organic compound is brought into contact with a heterogeneous catalyst during the reaction.

20. The process as claimed in claim 19, wherein the heterogeneous catalyst comprises the zeolite TS-1.

21. The process as claimed in claim 17, wherein the hydroperoxide used is hydrogen peroxide and the organic compound is brought into contact with a heterogeneous catalyst during the reaction and wherein the heterogeneous catalyst comprises the zeolite TS-1.

22. The process as claimed in claim 11, wherein the organic compound used is propylene and the oxirane is propylene oxide.

23. The process as claimed in claim 17, wherein the organic compound used is propylene and the oxirane is propylene oxide.

24. A continuously operated process for the intermediate isolation of propylene oxide formed by reaction of hydrogen peroxide with propylene in the propylene oxide synthesis, which process comprises at least the steps (i) to (iii):
(i) reacting the hydrogen peroxide with propylene to give a product mixture comprising the propylene oxide and unreacted hydrogen peroxide,
(ii) separating the unreacted hydrogen peroxide from the mixture resulting from step (i) by fractionating the mixture in a dividing wall column to give a low-boiling fraction, an intermediate-boiling fraction and a high boiling fraction, and the propylene oxide is taken off in the intermediate-boiling fraction at the side offtake and the hydrogen peroxide is taken off in the high-boiling fraction at the bottom of the column,
(iii) reacting the hydrogen peroxide which has been separated off in step (ii) with propylene,
with an isothermal fixed-bed reactor being used in step (i) and an adiabatic fixed-bed reactor being used in step (iii).

25. The process as claimed in claim 24, wherein the dividing wall column has from 10 to 70 theoretical plates and wherein the pressure at the top of the dividing wall column is from 0.5 to 5 bar and the distillation temperature at the side offtake is from 10 to 60° C.

26. The process as claimed in claim 24, wherein propylene is brought into contact with a heterogeneous catalyst during the reaction and wherein the heterogeneous catalyst comprises the zeolite TS-1.

27. The process as claimed in claim 24, wherein the sum of key components in the purified propylene oxide is less than 5% by weight, with the sum of propylene oxide and all the other components present in the propylene oxide being 100% by weight.

28. The process as claimed in claim 24, wherein the dividing wall column comprises at least two thermally coupled distillation columns.

29. A continuously operated process for the intermediate isolation of propylene oxide formed by reaction of hydrogen peroxide with propylene in the propylene oxide synthesis, which process comprises at least the steps (i) to (iii):
(i) reacting the hydrogen peroxide with propylene to give a product mixture comprising the propylene oxide and unreacted hydrogen peroxide, wherein propylene is brought into contact with a heterogeneous catalyst comprising the zeolite TS-1 during the reaction,
(ii) separating the unreacted hydrogen peroxide from the mixture resulting from step (i) by fractionating the mixture in a dividing wall column to give a low-boiling fraction, an intermediate-boiling fraction and a high-boiling fraction, and the propylene oxide is taken off in the intermediate-boiling fraction at the side offtake and the hydrogen peroxide is taken off in the high-boiling fraction at the bottom of the column, wherein the dividing wall column has from 10 to 70 theoretical plates and wherein the pressure at the top of the dividing wall column is from 0.5 to 5 bar and the distillation temperature at the side offtake is from 10 to 60° C.,
(iii) reacting the hydrogen peroxide which has been separated off in step (ii) with propylene, wherein propylene is brought into contact with a heterogeneous catalyst comprising the zeolite TS-1 during the reaction, and wherein the sum of key components in the purified propylene oxide is less than 5% by weight, with the sum of propylene oxide and all the other components present in the propylene oxide being 100% by weight,
with an isothermal fixed-bed reactor being used in step (i) and an adiabatic fixed-bed reactor being used in step (iii).

30. An apparatus for carrying out a continuously operated process for the intermediate isolation of the oxirane formed in the oxirane synthesis by reaction of a hydroperoxide with an organic compound, wherein the apparatus for preparing the oxirane comprises at least one isothermal reactor and one adiabatic reactor for carrying out the steps (i) and (iii) of a process comprising at least the steps (i) to (iii):
(i) reacting the hydroperoxide with the organic compound to give a product mixture comprising the reacted organic compound and unreacted hydroperoxide,
(ii) separating the unreacted hydroperoxide from the mixture,
(iii) reacting the hydroperoxide which has been separated off in step (ii) with the organic compound,
with an isothermal fixed-bed reactor being used in step (i) and an adiabatic fixed-bed reactor being used in step (iii), which apparatus further comprising a separation apparatus for the step (ii), where the separation apparatus comprises a dividing wall column having one or two side offtakes or at least two thermally coupled columns.
